(19) United States
(12) Patent Application Publication
(10) Pub. No.: US 2024/0157109 A1
(43) Pub. Date: May 16, 2024

(54) Connector Assemblies and Connecting Methods for Attaching Accessories to Luer Connectors

(71) Applicant: Becton, Dickinson and Company, Franklin Lakes, NJ (US)

(72) Inventors: Kevin M. Ryan, Whitehouse Station, NJ (US); Angela Marie Huenerfauth, Morris Plains, NJ (US)

(21) Appl. No.: 17/988,717

(22) Filed: Nov. 16, 2022

(51) Int. Cl. A61M 39/10; A61M 39/16

(52) U.S. Cl. CPC A61M 39/1011; A61M 39/162; A61M 2039/1072

(57) ABSTRACT

A connector assembly includes an inner connector having a first end, a second end, a sidewall extending between the first end and the second end, and a flange connected to or extending radially outward from the sidewall. The connector assembly also includes an outer connector configured to be inserted over the inner connector having a first end, a second end, a sidewall extending therebetween, and a lock extending radially inwardly from an inner surface of the sidewall configured to engage the flange of the inner connector, thereby securing the inner connector to the outer connector.

### BACKGROUND OF THE INVENTION

### Field of the Invention

The present disclosure relates generally to connector assemblies and connecting methods for attaching a first component or device, such as a fluid container or vascular access device, to a second component or device, such as an accessory device, and, in particular, to connector assemblies including universal attachment features configured to attach to devices with different types of connectors, such as either male luer connectors or female luer connectors.

### Description of Related Art

Medical devices, such as syringes, needle assemblies, perfusion devices, or transfusion devices, are commonly used for transferring and/or storing medical fluids. Such devices are often used for transferring medical fluids to the patient through one or more vascular access devices (VADs), which can include intravenous (IV) catheters, such as peripheral catheters or central venous catheters. VADs generally include access hubs, ports, or valves for connecting the VAD to the medical fluid supply, device, or container for transferring the medical fluid from the supply, device, or container to the patient through the VAD.

In many cases, needleless connector assemblies, interfaces, or arrangements are used for connecting the medical fluid supply, device, or container to the access hubs, ports, or valves of the VAD. One commonly used connection interface for connecting a medical fluid supply to a VAD, which is generally referred to as a luer connector, is governed by the International Organization for Standardization (ISO) 80369-7. As described in further detail herein, in conformity with the ISO standards, a male luer connector includes a conical distal tip having a taper of about 6% configured to be inserted into a tapered bore of a female luer. Such luer connecting assemblies or systems can provide a leak-free connections between the medical fluid supply, device, or container and the VAD. Further, such connection assemblies and systems are designed to provide protection against contamination of the medical fluids contained in the medical fluid supply, device, or container and passing through the VAD.

In some examples, a simple luer connector assembly or system can comprise male and female fittings that conform to the luer taper dimensions required by the ISO standards. To improve security and stability of the connection between the male fitting and the female fitting, locks can be provided, which are often referred to as luer-lock fittings. For example, an outer relief (e.g., helical threads) can be provided on the female fitting, which is configured to screw onto corresponding threads provided on an inner surface of a collar surrounding the male fitting.

In some cases, accessory devices are available that can be connected to male and/or female needleless or luer connectors. For example, such accessory devices can be attached to access hubs, ports, or valves of VADs before and/or after use to protect the VAD from contamination. Depending upon needs of particular clinical procedures or applications, the accessory devices can include, for example, disinfecting caps, safety components, and/or smart devices, such as devices comprising sensors for monitoring fluid flow through the VAD. For example, disinfecting caps can be disposable caps that contain an amount of cleaning or disinfecting solution for sterilizing portions of the port, hub, and valve of the VAD. An exemplary disinfecting cap is disclosed in U.S. Pat. No. 10,871,246, entitled “Universal Connector or Cap for Male and Female Threaded Fittings,” which is incorporated herein by reference in its entirety.

These disinfecting caps and other accessory devices are typically attached to the male or female luer connectors through a particular design of the accessory device that incorporates a thread capable of mating with corresponding luer threads of the luer connectors. However, access hubs and ports can have a variety of different types of male or female connectors for securing the hub or port to the VAD. Currently, practitioners often carry several types of disinfecting caps or other accessory devices with them, so that they can cap the different types of hubs and ports that may be used for a particular patient. For example, caps for male needleless connectors and female needleless connectors, as well as intravenous (IV) and hemodialysis lines, can use different connector designs and may require different caps or specific accessory devices. Therefore, a more universal connector interface is needed in order to more efficiently attach various accessory devices to medical fluid containers and VADs including male or female luer connectors.

### SUMMARY OF THE INVENTION

There can be significant drawbacks to the current methods for attaching accessory devices, such as disinfecting caps, to luer connectors of other devices, such as VADs. One drawback can be that there is not a robust way to attach the accessory device to the luer connector without using the luer threads of the needleless connector. However, when the accessory device is connected to the VAD via the luer threads, the accessory device may interfere with or prevent proper mating of the VAD to the medical fluid supply, device, or container (e.g., a syringe, IV line, or another container). Also, some accessory device are not intended to or do not need to be in contact with the threads or a fluid path of the VAD or medical fluid supply device. A second drawback is that an accessory device designed to engage threads of a VAD cannot mate onto both a male luer connector and a female luer connectors without having two sets of threads (i.e., both male threads and female threads). A third drawback can be that using multiple types of accessory devices can make sterilization more difficult and can significantly increase a number of product components that must be manufactured.

Accordingly, there is a need in the art for universal accessory attachment features that can be used with both male and female luer connectors. Desirably, the universal attachment features can be used to attach an accessory device to both male and female luer connectors of a VAD or medical fluid supply, device, or container. In particular, the universal attachment features desirably contain only a single thread or mating clip, which can be mated to devices comprising both male and female luer connectors. The connector assemblies, devices, and connecting methods disclosed herein are configured to address these drawbacks and issues.

According to an aspect of the disclosure, a connector assembly includes an inner connector having a first end, a second end, a sidewall extending between the first end and the second end, and a flange connected to or extending radially outward from the sidewall. The connector assembly also includes an outer connector configured to be inserted over the inner connector having a first end, a second end, a sidewall extending therebetween, and a lock extending radially inwardly from an inner surface of the sidewall configured to engage the flange of the inner connector, thereby securing the inner connector to the outer connector.

In accordance with an embodiment of the present invention, the inner connector includes a male luer connector.

In accordance with an embodiment of the present invention, the inner connector includes a stem having a proximal end connected to the sidewall, a distal end protruding distally beyond the second end of the sidewall, and a body extending between the proximal end and the distal end through a space defined by the sidewall of the inner connector; and at least one thread on an inner surface of the sidewall configured to engage corresponding threads of a female luer connector.

In accordance with an embodiment of the present invention, the stem includes a tapered outer surface.

In accordance with an embodiment of the present invention, the outer connector is configured to engage the inner connector without contacting the stem or the at least one thread of the inner connector.

In accordance with an embodiment of the present invention, the inner connector is configured to engage a female luer connector having a maximum outer diameter of less than 11.0 mm, or, preferably from about 9.0 mm to about 11.0 mm.

In accordance with an embodiment of the present invention, the inner connector is a female luer connector.

In accordance with an embodiment of the present invention, the sidewall of the inner connector includes threads on an outer surface of the sidewall configured to engage corresponding threads of a shield of a male luer connector.

In accordance with an embodiment of the present invention, the inner connector is sized so that a male luer connector having an outer diameter of from 8.0 mm to 14.0 mm or, preferably from about 9.0 mm to about 11.0 mm, can be inserted over the inner connector.

In accordance with an embodiment of the present invention, the outer connector is configured to engage the inner connector without contacting the threads on the outer surface of the sidewall of the inner connector.

In accordance with an embodiment of the present invention, the inner connector and/or the outer connector is a single-molded part integral with or attached to a vascular access device.

In accordance with an embodiment of the present invention, the inner connector and/or the outer connector includes a rigid thermoplastic polymer, such as polyester, polycarbonate, polypropylene, polyethylene, polyethylene terephthalate, or acrylonitrile butadiene styrene.

In accordance with an embodiment of the present invention, the inner connector includes a vascular access device and the outer connector includes an accessory device configured to engage the vascular access device.

In accordance with an embodiment of the present invention, the vascular access device includes at least one of a syringe, an infusion pump, a transfusion device, an intravenous drip device, or a catheter, and wherein the accessory device comprises a disinfecting cap, a safety device, a smart device, and/or a sensor device.

In accordance with an embodiment of the present invention, the outer connector includes a vascular access device and the inner connector includes an accessory device configured to engage the vascular access device.

In accordance with an embodiment of the present invention, the vascular access device includes at least one of a syringe, an infusion pump, a transfusion device, an intravenous drip device, or a catheter, and wherein the accessory device comprises a disinfecting cap, a safety device, a smart device, and/or a sensor device.

In accordance with an embodiment of the present invention, the flange connected to or extending radially outward from the sidewall of the inner connector comprises a peripheral edge comprising opposing flat portions and opposing curved portions between the flat portions.

In accordance with an embodiment of the present invention, the flange has a maximum outer diameter corresponding to an inner diameter of the sidewall of the outer connector.

In accordance with an embodiment of the present invention, the maximum outer diameter of the flange is about 8.0 mm to about 14.0 mm or, preferably, about 9.0 mm to about 11.0 mm.

In accordance with an embodiment of the present invention, the flange has a thickness of from 0.2 mm to 2 mm.

In accordance with an embodiment of the present invention, the flange is disposed on or connected to the second end of the inner connector.

In accordance with an embodiment of the present invention, the flange is disposed on the sidewall of the inner connector between the first end and the second end of the inner connector.

In accordance with an embodiment of the present invention, the lock includes a thread extending radially inwardly from at least a portion of the inner surface of the sidewall of the outer connector.

In accordance with an embodiment of the present invention, the thread is configured to rotatably engage the flange of the inner connector.

In accordance with an embodiment of the present invention, the thread extends about the inner surface of the sidewall of the outer connector by less than a full rotation.

In accordance with an embodiment of the present invention, the flange of the inner connector includes a peripheral edge comprising opposing flat portions and opposing curved portions between the flat portions, wherein the lock comprises a thread extending radially inwardly from at least a portion of the inner surface of the sidewall of the outer connector, and wherein the outer connector further comprises at least one blocking tab separate from the thread extending radially inward from the sidewall of the outer connector disposed at the first end of the outer connector configured to contact the opposing curved portions of the flange to block further advancement of the inner connector into the outer connector.

In accordance with an embodiment of the present invention, the at least one blocking tab includes a pair of opposed blocking tabs positioned to contact the opposing curved portions of the peripheral edge of the flange when the outer connector is fully connected to the inner connector.

In accordance with an embodiment of the present invention, the lock includes at least one snap tab configured to form a snap-fit engagement with the flange of the inner connector.

In accordance with an embodiment of the present invention, the lock includes a locking ring having an annular sidewall, a plurality of first tabs extending radially inwardly from a first annular edge of the sidewall and a plurality of second tabs extending radially inwardly from a second annular edge of the sidewall.

In accordance with an embodiment of the present invention, the plurality of first tabs and the plurality of second tabs are configured to retain the flange of the inner connector by a snap-fit engagement.

In accordance with an embodiment of the present invention, the plurality of first tabs and the plurality of second tabs are configured to retain the flange of the inner connector within a space between the first tabs and the second tabs.

In accordance with an embodiment of the present invention, the plurality of second tabs comprise chamfered surfaces for facilitating insertion of the inner connector into the outer connector.

In accordance with an embodiment of the present invention, the flange is inserted into the locking ring by applying an axially directed force to the inner connector sufficient to deflect the plurality of second tabs away from the flange so that the flange moves into a space between the first tabs and the second tabs.

In accordance with an embodiment of the present invention, the flange is removed from the locking ring by applying a sufficient axial force to the inner connector or to the outer connector to overcome the snap-fit engagement between the locking ring and the flange so that the inner connector can be moved axially relative to the outer connector.

In accordance with an embodiment of the present invention, a distance between the first tabs and the second tabs corresponds to a thickness of the flange of the inner connector.

According to another aspect of the disclosure, a vascular access device configured to be connected to an accessory device includes a tubular body defining a fluid path extending at least partially through the tubular body including a proximal end portion and a distal end portion. The vascular access device also includes a male connector connected to the tubular body including: a stem defining a fluid path fluidly connected to the distal end portion of the fluid path of the tubular body configured to be inserted into a fluid path of a female connector; a shield connected to a first end of the stem and at least partially enclosing the stem; and a thread extending radially inward from an inner surface of the shield configured to engage a corresponding thread on an outer surface of the female connector. The vascular access device also includes a flange connected to or extending radially outward from a sidewall of the shield configured to engage a lock extending radially inward from an inner surface of a tubular sidewall of the accessory device.

In accordance with an embodiment of the present invention, the stem includes a tapered outer surface.

In accordance with an embodiment of the present invention, the accessory device is configured to engage the flange of the vascular access device without contacting the stem or the at least one thread.

In accordance with an embodiment of the present invention, the vascular access device includes at least one of a syringe, an infusion pump, a transfusion device, an intravenous drip device, or a catheter, and wherein the accessory device comprises a disinfecting cap, a safety device, a smart device, and/or a sensor device.

In accordance with an embodiment of the present invention, the flange includes a peripheral edge comprising opposing flat portions and opposing curved portions between the flat portions.

According to another aspect of the disclosure, an attachment method for attaching an inner connector to an outer connector includes a step of inserting an inner connector having a first end, a second end, a sidewall extending between the first end and the second end, and a flange connected to or extending radially outward from the sidewall, into an outer connector. The outer connector includes a lock extending radially inwardly from an inner surface of the outer connector. The method also includes a step of moving the inner connector through the outer connector, thereby causing the flange of the inner connector to engage the lock of the outer connector to secure the inner connector to the outer connector.

In accordance with an embodiment of the present invention, the inner connector includes a male luer connector or a female luer connector.

In accordance with an embodiment of the present invention, the inner connector includes a vascular access device and the outer connector comprises an accessory device configured to engage the vascular access device.

In accordance with an embodiment of the present invention, the vascular access device includes at least one of a syringe, an infusion pump, a transfusion device, an intravenous drip device, or a catheter, and wherein the accessory device includes a disinfecting cap, a safety device, a smart device, and/or a sensor device.

In accordance with an embodiment of the present invention, the lock includes a thread extending radially inwardly from at least a portion of the inner surface of the outer connector, and wherein inserting the inner connector into the outer connector comprises pressing the flange of the inner connector against the thread of the outer connector.

In accordance with an embodiment of the present invention, wherein moving the inner connector through the outer connector includes rotating the inner connector relative to the outer connector to draw the flange along the thread of the outer connector.

In accordance with an embodiment of the present invention, the lock includes a locking ring including an annular sidewall, a plurality of first tabs extending radially inwardly from a first annular edge of the sidewall and a plurality of second tabs extending radially inwardly from a second annular edge of the sidewall, and wherein moving the inner connector through the outer connector comprises pressing the flange against the plurality of second tabs with sufficient force to deflect the second tabs away from the flange.

### BRIEF DESCRIPTION OF THE DRAWINGS

FIG. 1A is a cross-sectional view of an exemplary male connector, as is known in the prior art.

FIG. 1B is an example of a female connector including a septum with a slit, as is known in the prior art.

FIG. 2A is a perspective view of an inner connector comprising a flange configured to be engaged to a lock of an outer connector, according to an aspect of the present disclosure.

FIG. 2B is a cross-sectional view of the inner connector of FIG. 2A.

FIG. 2C is a front view of a syringe including the inner connector of FIG. 2A, according to an aspect of the present disclosure.

FIG. 3A is a perspective view of another example of an inner connector comprising a flange configured to be engaged to a lock of an outer connector, according to an aspect of the present disclosure.

FIG. 3B is a cross-sectional view of the inner connector of FIG. 3A.

FIG. 3C is a front view of a syringe including the inner connector of FIG. 3A, according to an aspect of the present disclosure.

FIG. 4A is a perspective view of a locking ring of an outer connector, according to an aspect of the present disclosure.

FIG. 4B is a bottom view of the locking ring of FIG. 4A.

FIG. 4C is a cross-sectional view of an accessory device including the locking ring of FIG. 4A, according to an aspect of the present disclosure.

FIG. 5A is a cross-sectional view of a syringe with an inner connector mounted to an accessory device including a locking ring, according to an aspect of the present disclosure.

FIG. 5B is a cross-sectional view of a syringe with another example of an inner connector mounted to an accessory device including a locking ring, according to an aspect of the present disclosure.

FIG. 6 is a flow chart showing a method for connecting an inner connector to an outer connector, according to an aspect of the present disclosure.

FIG. 7A is a perspective view of another example of an outer connector including a snap ring, according to an aspect of the present disclosure.

FIG. 7B is a cross-sectional view of the outer connector of FIG. 7A.

FIG. 8A is a cross-sectional view of a syringe with an inner connector mounted to an accessory device including an outer connector with a snap ring, according to an aspect of the disclosure.

FIG. 8B is a cross-sectional view of a syringe with another example of an inner connector mounted to an accessory device including an outer connector with a snap ring, according to an aspect of the disclosure.

### DESCRIPTION OF THE INVENTION

The following description is provided to enable those skilled in the art to make and use the described embodiments contemplated for carrying out the invention. Various modifications, equivalents, variations, and alternatives, however, will remain readily apparent to those skilled in the art. Any and all such modifications, variations, equivalents, and alternatives are intended to fall within the spirit and scope of the present invention.

For purposes of the description hereinafter, the terms “upper”, “lower”, “right”, “left”, “vertical”, “horizontal”, “top”, “bottom”, “lateral”, “longitudinal”, and derivatives thereof shall relate to the invention as it is oriented in the drawing figures. The term “proximal” refers to a portion of a device or part that is grasped by a user or connected to another device or part. The term “distal” refers to a portion of a device or part that is opposite the proximal portion (i.e., farthest away from the portion that is grasped by a user or connected to another part). For example, for an implantable medical device, such as a catheter, a proximal portion can refer to the portion of the catheter that remains outside of a patient's body and is manipulated by a user. The distal portion of the catheter can be the portion that is inserted into the vasculature of the patient. For an object or part, such as an elongated member, the proximal end can be the end that is connected to another object or part. The distal end of the elongated member is opposite the proximal end and can be free from connections to other objects or parts. However, it is to be understood that the invention may assume alternative variations and step sequences, except where expressly specified to the contrary. It is also to be understood that the specific devices and processes illustrated in the attached drawings, and described in the following specification, are simply exemplary embodiments of the invention. Hence, specific dimensions and other physical characteristics related to the embodiments disclosed herein are not to be considered as limiting.

The present disclosure is directed to a connector assembly 10 or connector interface that allows a practitioner, such as a clinician or healthcare worker, to connect a first component or device to a second component or device. As used herein, the “healthcare worker” can be a medical professional, such as a medical technician or nurse, trained to perform a medical procedure, such as a fluid delivery or blood collection procedure. As previously described, the first component or device can be a medical fluid supply, device, or container, such as a syringe barrel, needle assembly, perfusion device, or transfusion device. The first component or device can also be a port, hub, or valve of a vascular access device (VADs). The second component or device can be an accessory device, such as a disinfecting cap, safety device, or smart device, such as a sensor configured to monitor fluid flow.

The first component and the second component can each include male or female luer connectors or fittings. However, the connector assembly 10 or interface disclosed herein connects the first component or device to the second component or device without using the male or female luer connectors or fittings. Instead, as described in further detail herein, the first component or device is connected to the second component or device by a connection or engagement between a radially extending flange on an inner connector 12 and a lock or locking ring on an outer connector 14 that connects to the flange by, for example, a threaded or snap-fit engagement. In this way, the first component or device can be joined to the second component or device without contacting, contaminating, or otherwise deforming threads or other parts of the male or female luer connectors or fittings. In some examples, the inner connector 12 and the outer connector 14 can be configured to remain attached together permanently. In other examples, the inner connector 12 can be configured to remain removably connected to the outer connector 14 for predetermined period, such as for at least seven days, which is a maximum time of recommended use for disinfecting caps permitted by many medical facility sterile practice guidelines. The inner connector 12 and outer connector 14 disclosed herein can be configured to join various male and female connectors having different dimensions, sizes, or thread profiles. Therefore, the connection assemblies 10 disclosed herein can be universal connector assemblies for forming temporary, removable, or permanent engagements between various types of medical fluid containers or VADs and accessory devices.

### Male and Female Luer Connectors

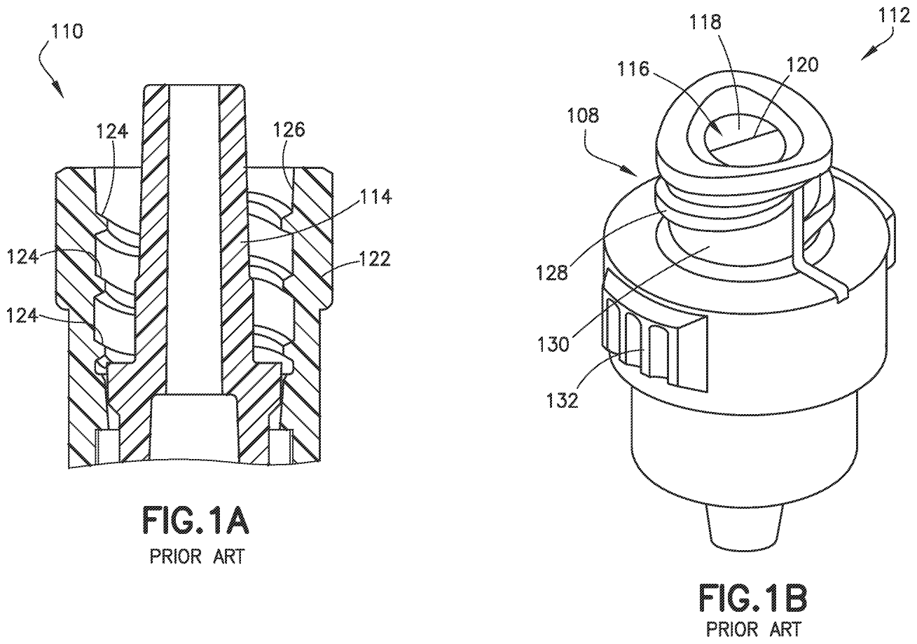

As previously described, the medical fluid devices, VADs, and accessory devices described herein can include various types and sizes of male or female luer connectors. As used herein, a “male connector” refers to a connector 110 comprising an elongated member, such as a tubular member or stem 114, configured to be inserted in a bore, tube, or opening having an inner diameter that is larger than an outermost diameter of the male connector 110. An exemplary male connector 110 is shown in FIG. 1A. A “female connector” refers to a connector 112 comprising an opening or port 116 that is configured to receive an elongated member or tubular member of another object or device in order to connect the object or device to the female connector 112. The female connector 112 can have a cover or septum 118 over the opening 116. An exemplary female connector 112 including a septum 118 with a slit 120 is shown in FIG. 1B.

As used herein, a “luer connector” refers to a connector that includes a tapered portion (e.g., a luer taper having a taper of about 6%) for creating a friction engagement between a tapered stem 114 or elongated member of the male luer connector 110 and a tapered cavity. Specifically, the male luer connector 110 can include a tapered stem 114 or elongated member having a tapered outer surface. The female luer connector 112 can include a tapered bore or cavity (e.g., a cavity tapered by about 6%) configured to receive and engage the tapered stem 114 or elongated member to connect the male luer connector 110 to the female luer connector 112.

In order to secure the male and female connectors 110, 112 together, in some examples, the connectors 110, 112 can include engaging structures, such as threads, for drawing the connectors 110, 112 together. For example, as shown in FIG. 1A, the male luer connector 110 can include an annular shield 122 having an outer diameter of about 9 mm to about 11 mm extending about the tapered stem 114. More particularly, many commercially available male connectors 110 have an annular shield 122 with an outer diameter of either 9.1 mm to 9.4 mm or 10.3 mm to 10.4 mm. For example, male connectors 110 by RyMed, B. Braun, Zyno, and Smith's Medical often have an outer diameter of about 9.1 mm to 9.4 mm. By contrast, male connectors 110 by Kendall, Becton Dickinson, ICU Medicals, Baxter, and Kawasumi often have an outer diameter of about 10.4 mm. The connecting assemblies 10 disclosed herein can be configured to engage and/or form connections with any of these commercially available male connectors 110.

As shown in FIG. 1A, the annular shield 122 can include threads 124 on an inner surface 126 of the shield 122 configured to engage corresponding threads 128 on an outer surface 130 of the female luer connector 112. As shown in FIG. 1B, the female luer connector 112 can include threads 128 extending from the outer surface 130 positioned to engage the threads 124 on the inner surface 126 of the annular shield 122 of the male luer connector 110. Twisting the female connector 112 relative to the male connector 110 causes the corresponding threads 124, 128 to engage, which draws the connectors 110, 112 together, such that the tapered stem 114 or elongated member of the male luer connector 110 moves through the opening 116 of the female connector 112. In some examples, the female connector 112 can also include vertical ridges 132 near a proximal end of the female connector 112, which can be used to manipulate the female connector 112 making it easier to twist the female connector 112 compared to another connector or device.

There are numerous different commercially available medical devices, such as hubs, ports, and valves, which can include different variations of male or female connectors 110, 112. As previously described, any of these commercially available connectors can be modified or adapted to work with the connector assemblies 10 of the present disclosure. For example, the connecting assemblies 10 of the present disclosure can be configured to attach to male or female Luer-Lok™ connectors by Becton Dickinson and Company. The connector assemblies 10 of the present disclosure can also be configured to connect to different connector designs including, without limitation, the BD Q-Syte™, BD MaxZero™, BD MaxPlus™, and SmartSite™ needle free connectors by Becton Dickinson and Company. The connecting assemblies 10 can also be used with male and/or female connectors by other manufactures including, without limitation, MicroClave® connectors (ICU Medical Inc.) and Ultrasite® connectors (B. Braun Medical Inc.). In other examples, the connecting assemblies can be configured for use with one or more of the following commercially available male connectors: Kendall 2001NP; BD MP5303-C; ICU Med 12664-28; RyMed RYM-5307HPU; B. Braun 470108; Baxter 2C8537; Kawasumi IV-0094; Zyno B2-70071-D; B. Braun 470124; Baxter 2C7462; and Smith's Medical 536035.

### Connector Assembly Including a Threaded Locking Ring

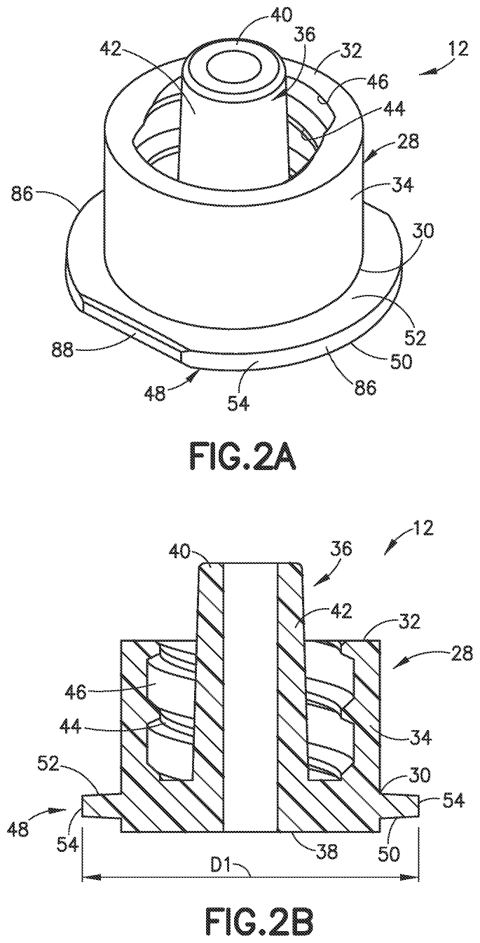

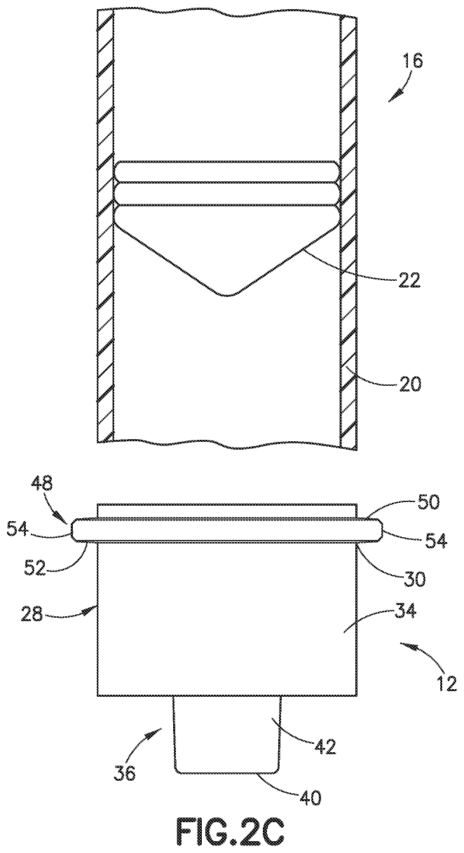

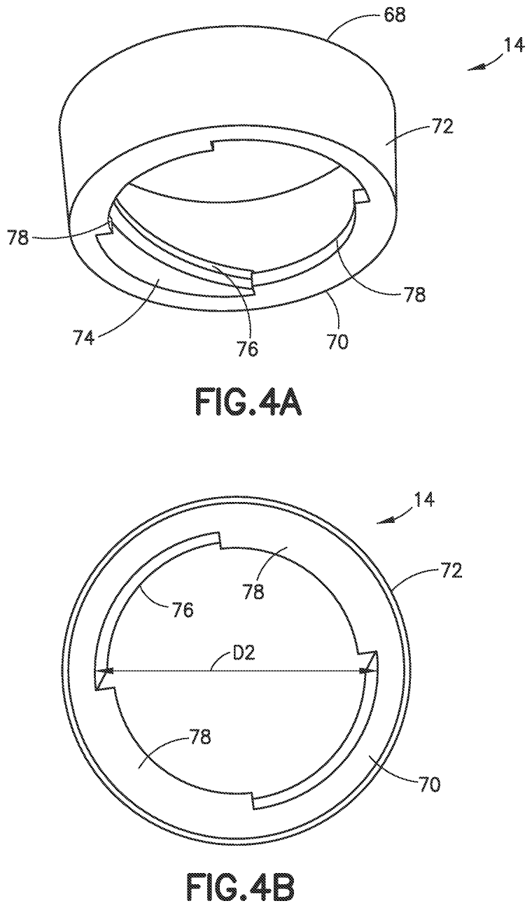

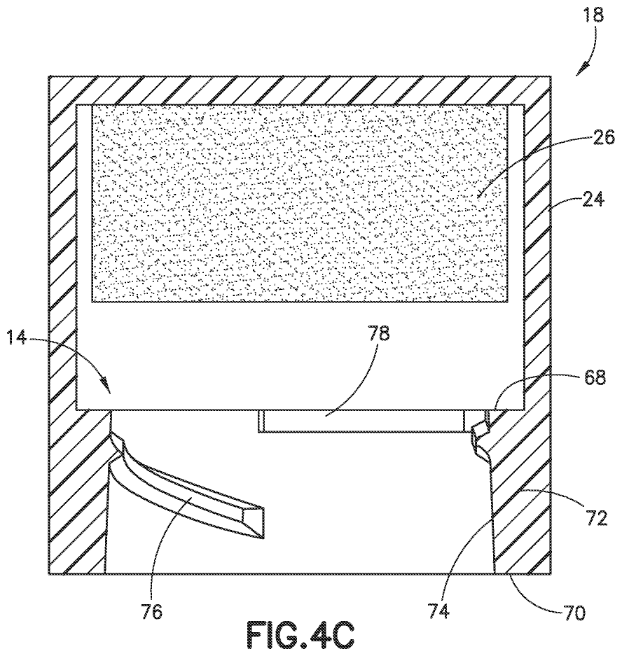

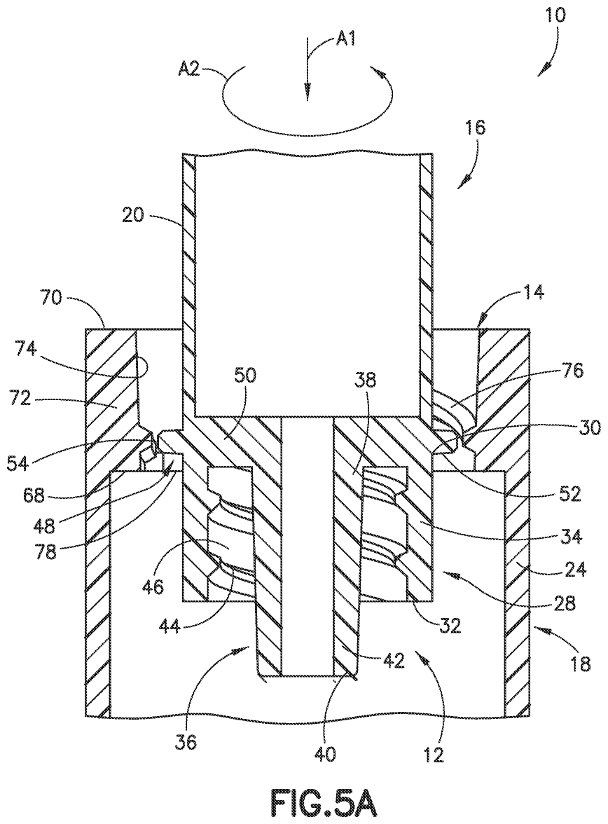

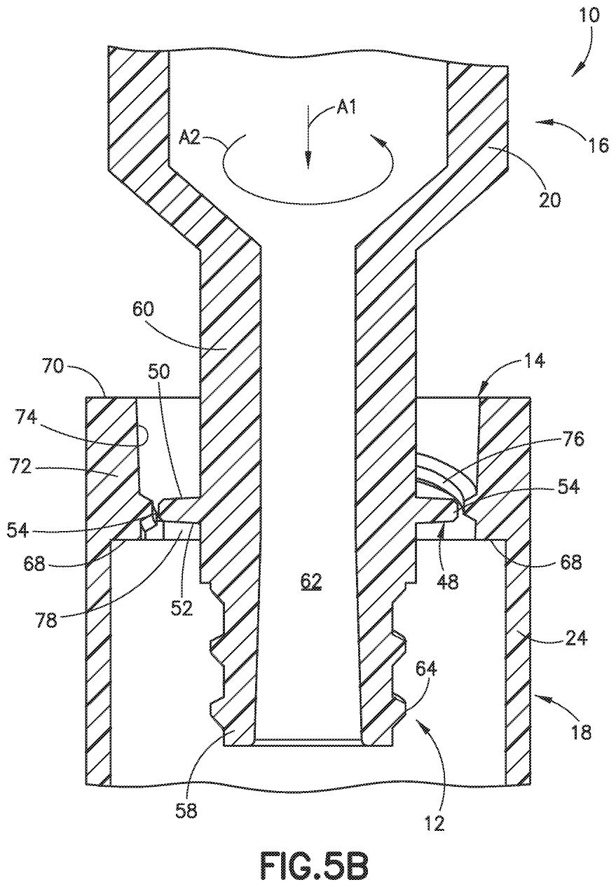

FIGS. 2A-5B show aspects of a connector assembly 10 or connector interface for connecting a medical fluid container, such as a syringe 16 (shown in FIGS. 2C, 3C, 5A, and 5B), to an accessory device, such as the disinfecting cap 18 (shown in FIGS. 4C, 5A, and 5B).

In some examples, the connector assembly 10 comprises the inner connector 12 that is configured to be inserted into and engaged with the outer connector 14. As shown in FIGS. 2C, 3C, 5A, and 5B, the inner connector 12 can be integral with or connected to a medical fluid container, such as the syringe 16, which, as shown in FIG. 2C, comprises a barrel 20 and a plunger or stopper 22 positioned in the barrel 20. The inner connector 12 can also be integral with or connected to components of a VAD, such as an access hub, port, or valve of the VAD. As shown in FIGS. 4C, 5A, and 5B, the outer connector 14 can be integral with or connected to the accessory device, such as the disinfecting cap 18, which, as shown in FIG. 4C, comprises a housing 24 enclosing an absorbent member or sponge 26 containing a disinfecting or cleaning solution.

More specifically, as shown in FIG. 4C, the sponge 26 can be a cylindrical structure or member received within the housing 24. The sponge 26 can be formed from an absorbent material capable of absorbing a cleaning or disinfecting solution for cleaning and/or disinfecting portions of the male connector 110 and the female connector 112 (shown in FIGS. 1A and 1B). The sponge 26 can comprise a thermoplastic elastomer, such as polypropylene, polyethylene, or synthetic or natural rubber (e.g., isoprene). The sponge 26 can be provided (i.e., presoaked) with the cleaning or disinfecting solution. For example, the cleaning or disinfecting solution can be an antimicrobial, anti-fungal, antibacterial, or antiviral solution that cleans and sterilizes surfaces of the connectors 110, 112. In some examples, the cleaning solution can be isopropyl alcohol (IPA), such as about 70% isopropyl alcohol (IPA). In other examples, the cleaning solution can be about 0.5% to about 3.5% chlorhexidine gluconate alone or in combination with about 70% IPA.

In other examples, while not shown in the present figures, the inner connector 12 can be connected to or integral with the accessory device, such as the disinfecting cap 18, and the outer connector 14 can be integral with or connected to the medical fluid container, such as the syringe 16, or the VAD.

Generally, the inner connector 12 and/or the outer connector 14 are integral parts formed by a single molding process, such as by a single injection molding process. The inner connector 12 and/or the outer connector 14 can be formed from or comprise a rigid material, such as a rigid thermoplastic polymer (e.g., polyester, polycarbonate, polypropylene, polyethylene, polyethylene terephthalate, or acrylonitrile butadiene styrene). In some cases, the inner connector 12 and/or the outer connector 14 are also integral with parts of the medical fluid container, VAD, or accessory device. In other examples, the inner connector 12 or the outer connector 14 can be separate parts that are mounted to the medical fluid container, VAD, or accessory device by adhesives or mechanical fasteners, as are known in the art.

As shown in FIGS. 2A-2C, the inner connector 12 can comprise components of a male luer connector, such as components of the male connector 110 shown in FIG. 1A. For example, the inner connector 12 can include a tubular body or shield 28 comprising a first or proximal end 30, a second or distal end 32, and a sidewall 34 extending therebetween. The shield 28 can be sized to receive a tubular portion of a female luer connector, such as the female luer connector 112 shown in FIG. 1B, having an outer diameter of less than 11.0 mm, or, preferably from about 9.0 mm to about 11.0 mm. The inner connector 12 further comprises a stem 36 defining a fluid path through the inner connector 12. The stem 36 comprises a proximal end 38 connected to the proximal end 30 of the shield 28, a distal end 40 that extends axially beyond the distal end 32 of the shield 28, and a sidewall or body 42 extending between the proximal end 38 and the distal end 40 through a space defined by the sidewall 34 of the shield 28. As previously described, the stem 36 can include a tapered outer surface configured to be inserted into a tapered cavity or bore of a female luer connector. In some examples, the shield 28 also includes a thread 44, such as a helical thread, extending radially inward from an inner surface 46 of the shield 28. As shown in FIGS. 2A-2C, the inner connector 12 further comprises a flange 48 extending radially outward from the shield 28.

The flange 48 is a generally disk-shaped structure comprising a substantially flat proximal surface 50, an opposing substantially flat distal surface 52, and a peripheral edge 54 extending between the proximal surface 50 and the distal surface 52. The peripheral edge 54 can include opposing flat portions and opposing curved portions 86 between the flat portions 88. Dimensions of the flange 48 are selected so that the flange 48 can be inserted into the outer connector 14 for engaging the inner connector 12 to the outer connector 14. For example, the flange 48 can have a maximum outer diameter D1 (shown in FIG. 2B) corresponding to an inner diameter D2 (shown in FIG. 4B) of the outer connecter 14. For example, the maximum outer diameter D1 can be from about 6.2 mm to about 6.8 mm. The flange 48 can also have a maximum thickness, defined as the maximum axial distance between the proximal surface 50 and the distal surface 52, of from about 0.2 mm to about 1.0 mm.

The flange 48 can be disposed at various positions on the outer surface of the sidewall or shield 28. For example, as shown in FIGS. 2A-2C, the flange 48 is positioned proximate to the proximal end 30 of the shield 28. In other examples, the flange 48 can be positioned at a midpoint of the shield 28 halfway between the proximal end 30 of the shield 28 and the distal end 32 of the shield 28. In other examples, the flange 48 can be positioned closer to the distal end 32 of the shield 28 than the proximal end 30 or at any other convenient location depending upon the design of the syringe, VAD, and/or accessory device.

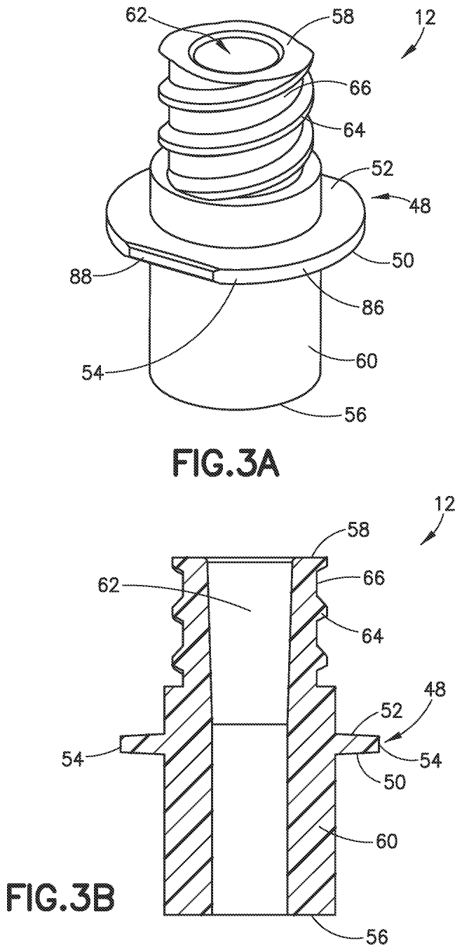

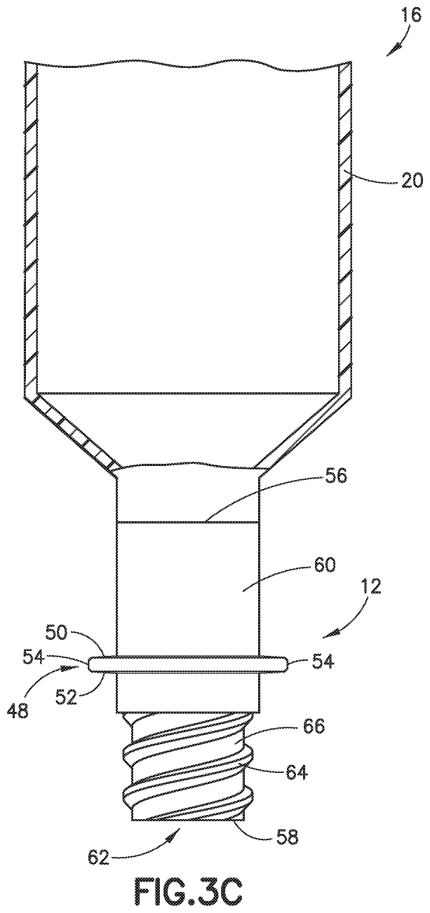

As shown in FIGS. 3A-3C, the inner connector 12 can alternatively comprise components of a female luer connector, such as components of the female luer connector 112 shown in FIG. 1B. For example, the inner connector 12 can include a tubular body comprising a first or proximal end 56, a second or distal end 58, and a sidewall 60 extending therebetween. As shown in FIG. 3B, the sidewall 60 of the tubular body encloses a central channel 62. A distal-most portion of the central channel 62 is tapered with a taper corresponding to a taper of an outer surface of a stem 114 of a male connector 110 (as shown in FIG. 1A). The distal-most portion of the inner connector 12 also includes a thread 64, such as a helical thread, on an outer surface 66 of the sidewall 60 configured to engage corresponding threads of a shield 122 of a male luer connector 110 (shown in FIG. 1A). For example, the threads 64 of the inner connector 12 can be sized so that a male luer connector 110 having an outer diameter of from 8.0 mm to 14.0 mm or, preferably, from about 9.0 mm to about 11.0 mm, can be inserted over the inner connector 12. The inner connector 12 shown in FIGS. 3A-3C also includes the flange 48 extending radially outward from the sidewall 60. The flange 48 of FIGS. 3A-3C is generally identical to previously described examples, including the substantially flat proximal surface 50, the opposing distal surface 52, and the peripheral edge 54. As in previous examples, the peripheral edge 54 can include the curved portions 86 and the flat portions 88.

As shown in FIGS. 3A-3C, the flange 48 is disposed at an intermediate position between the proximal end 56 and the distal end 58 of the inner connector 12. Also, the flange 48 is positioned proximal to the threads 64 extending about the distal-most portion of the sidewall 60 of the inner connector 12. As shown in FIG. 3C, the inner connector 12 including the components of the female luer connector is integral with the syringe barrel 20, such that fluid in the syringe barrel 20 passes through a distal open end of the barrel 20 and into the channel 62 of the inner connector 12.

The connector assembly 10 of the present disclosure also includes the outer connector 14 configured to engage the inner connector 12. For example, the outer connector 14 can be configured to be inserted over the inner connector 12 for temporarily or permanently fixing the outer connector 14 to the inner connector 12. As shown in FIGS. 4A and 4B, the outer connector 14 can be an annular body or ring comprising a first or proximal end 68, a second or distal end 70, and a sidewall 72 extending between the proximal end 68 and the distal end 70. The outer connector 14 also includes a protrusion, latch, lock, or similar engaging structure extending radially inward from an inner surface 74 of the sidewall 70 for engaging the flange 48 of the inner connector 12 in order to secure the inner connector 12 to the outer connector 14.

In some examples, the engaging structure or lock can comprise a thread 76 extending radially inward from the inner surface 74 of the sidewall 72 of the outer connector 14. The thread 76 of the outer connector 14 can be configured to rotatably engage the flange 48 of the inner connector 12. The thread 48 can be provided in a variety of different configurations depending upon the design of the inner connector 12 and/or devices or components connected to or integral with the inner connector 12. For example, as shown in FIGS. 4A-4C, the thread 76 can extend about the inner surface 74 of the sidewall 72 of the outer connector 14 by less than a full rotation. In this configuration, the practitioner need only rotate the outer connector 14 relative to the inner connector 12 by less than a full rotation to fully engage the inner connecter 12 within the outer connector 14. In other examples, the thread 76 can extend about the inner surface 74 of the sidewall 72 by a full rotation, two full rotations, or any other convenient amount. Generally including additional rotations means that the inner connector 12 is more securely engaged to the outer connector 14 making it less likely that the inner connector 12 will be removed from the outer connector 14 at an unexpected or inconvenient time.

The outer connector 14 can also include one or more blocking tabs 78, which are separate from the thread 76, extending radially inward from the inner surface 74 of the sidewall 72. The blocking tabs 78 are disposed at the proximal end 68 of the outer connector 14 and are configured to block further advancement of the inner connector 12 into the outer connector 14. For example, as shown most clearly in FIG. 4B, the outer connector 14 can comprise a pair of opposed blocking tabs 78 positioned to contact the opposing curved portions 86 of the peripheral edge 54 of the flange 48 when the outer connector 14 is fully connected to the inner connector 12. More specifically, as shown in FIGS. 5A and 5B, the blocking tabs 78 are positioned so that the distal surface 52 of the flange 48 rests against the blocking tabs 78, when the inner connector 12 is fully inserted into the outer connector 14. The blocking tabs 78 prevent the practitioner from over tightening the connectors 12, 14 ensuring that the inner connector 12 is correctly positioned relative to the outer connector 14 and can be easily removed from the outer 14 at an appropriate time.

As shown in FIG. 4C, the outer connector 14 can be integral with or connected to the accessory device, such as the disinfecting cap 18. As previously described, the disinfecting cap 18 comprises the housing 24, which comprises a closed end, an open end, and a sidewall extending between the closed end and the open end. The disinfecting cap 18 also includes the sponge 26 positioned in the housing 24. The outer connector 14 is positioned proximate to the open end of the housing 24. In some examples, the outer connector 14 can be integral with the housing 24 of the disinfecting cap 18. Alternatively, the outer connector 14 can be attached to the housing 24 by an adhesive or mechanical fastener, as are known in the art.

As previously described, the inner connector 12 is configured to be removably engaged to the outer connector 14 by a rotational engagement between the flange 48 of the inner connector 12 and the threads 76 of the outer connector 14. FIGS. 5A and 5B are cross-sectional views showing the syringe 16 comprising the inner connector 12 connected to the disinfecting cap 18 comprising the outer connector 14. Specifically, as shown in FIG. 5A, the inner connector 12 includes the components of a male luer connector, specifically the shield 28 and the stem 36. The shield 28 and stem 36 are positioned within and enclosed by the housing 24 of the disinfecting cap 18, thereby protecting the shield 28 and stem 36 from contacting microbes, particles, dirt, dust, and other debris that could contaminate the male luer connector components or syringe barrel 20 connected thereto. Significantly, the outer connector 14 is engaged to the inner connector 12 without contacting the stem 36, shield 28, or threads 44 of the inner connector. Instead, as shown in FIG. 5A, the flange 48 is engaged to the threads 76 of the outer connector 14 and rests against the blocking tabs 78 of the outer connector 14. Accordingly, the inner connector 12 is engaged to the outer connector 14 without contacting or using any of the components of the male luer connector, meaning that the outer connector 14 does not contaminate, deform, or otherwise damage these components of the male luer connector. As such, these components are preserved to be used for connecting the male luer connector to a female needless connector when, for example, the syringe 16 is in use after the disinfecting cap 18 has been removed.

FIG. 5B shows the inner connector 12 including features of a female luer connector connected to the outer connector 14 including features of the disinfecting cap 18. As shown in FIG. 5B, the flange 48 of the inner connector 12 is engaged with the threads 76 of the outer connector 14 and rests against the blocking tab 78 of the outer connector 14. Also, the distal-most portion of the inner connector 12 including the threads 64 on the outer surface 66 of the inner connector 12 are enclosed within the housing 24 of the disinfecting cap 18. Accordingly, the threads 64 of the inner connector 12 are not used for attaching the inner connector 12 to the disinfecting cap 18. Also, no portion of the disinfecting cap 18 is inserted into the central channel 62 of the inner connector 12. Accordingly, these structures of the inner connector 12, which are used for attaching the inner connector 12 to a male luer connector after the disinfecting cap 18 is removed, are not contaminated, deformed, or otherwise contacted by the outer connector 14.

### Methods for Attaching an Accessory Device to a Luer Connector

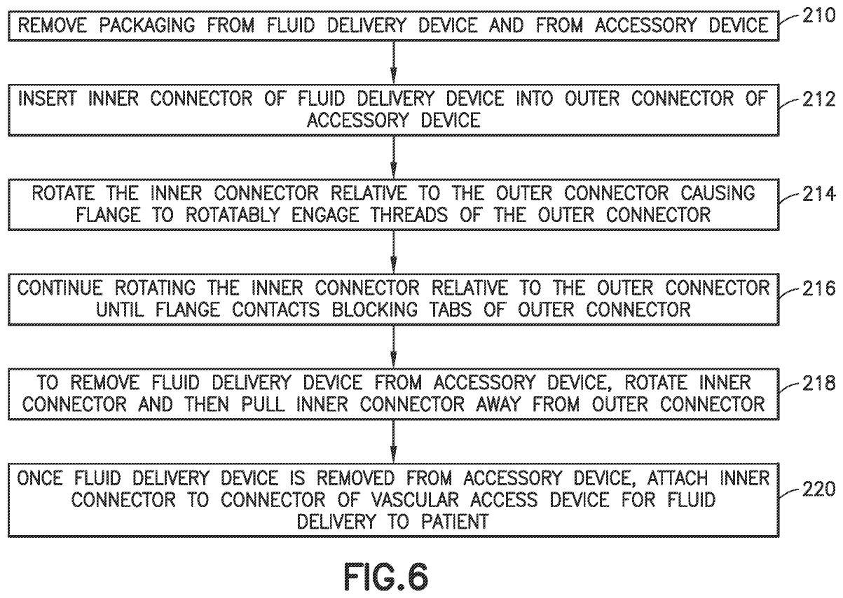

As previously described, the connector assemblies 10 of the present disclosure are configured to be used for connecting together devices and components of a variety of sizes, structures, and designs. The devices can include components of either male luer connectors or female luer connectors. FIG. 6 is a flow chart showing steps for connecting the inner connector 12 to the outer connector 14

In order to connect a device, such as a syringe 16, comprising the inner connector 12 to an accessory device, such as the disinfecting cap 18, comprising the outer connector 14, at step 210, the practitioner first removes any packaging from the syringe 16 and disinfecting cap 18. For example, the practitioner may remove a protective cover or film from the open end of the housing 24 of the disinfecting cap 18. The practitioner may also remove any caps, covers, sheaths, or other packaging from the syringe 16.

At step 212, the practitioner then pushes the inner connector 12 in a direction of arrow A1 (shown in FIGS. 5A and 5B) into the outer connector 18 in order to bring the flange 48 of the inner connector 12 into contact with the threads 76 on the inner surface 74 of the outer connector 14.

At step 214, once the flange 48 is in contact with the threads 76, the practitioner rotates the inner connector 12 relative to the outer connector 14, as shown by arrow A2 (in FIGS. 5A and 5B). Rotation of the inner connector 12 relative to the outer connector 14 causes the flange 48 to move along the threads 76 drawing the inner connector 12 farther into the outer connector 14.

At step 216, the practitioner continues to rotate the inner connector 12 relative to the outer connector 14 until the distal surface 52 of the flange 48 rests against the blocking tabs 78 of the outer connector 14, as shown in FIGS. 5A and 5B. As previously described, when the flange 48 is in contact with the blocking tabs 78, the inner connector 12 is fully engaged to the outer connector 14. The blocking tabs 78 prevent further axial advancement of the inner connector 12 relative to the outer connector 14.

In this position, as shown in FIG. 5A, the stem 36 and shield 28 of the inner connector 12 are enclosed within the housing 24 of the disinfecting cap 18. Also, no portion of the outer connector 14 or disinfecting cap 18 engages or contacts the stem 36 or the threads 44 on the shield 28 of the inner connector 12. In a similar manner, for the inner connector 12 comprising components of a female luer connector shown in FIG. 5B, the distal-most portion of the inner connector 12, which includes the threads 64 on the outer surface 66 of the inner connector 12, is fully enclosed within the housing 24 of the disinfecting cap 18. Also, no portion of the outer connector 14 or disinfecting cap 18 is engaged with the outer threads 64 of the inner connector 12 or inserted into the tapered central cavity 62 or bore of the inner connector 12.

At step 218, in order to remove the inner connector 12 from the outer connector 14, the practitioner rotates the inner connector 12 relative to the outer connector 14 in a direction opposite from arrow A2 (shown in FIGS. 5A and 5B) causing the flange 48 to move away from or back off from the threads 76 of the outer connector 14. Once the flange 48 is released from the threads 76, the practitioner can move the inner connector 12 and syringe 16 away from the outer connector 14 and disinfecting cap 18.

At step 220, once the inner connector 12 and syringe 16 are removed from the outer connector 14 and disinfecting cap 18, the inner connector 12 can be connected to another accessory device or to a VAD. In particular, the VAD can be used for providing the medical fluid from the syringe barrel 20 to the patient through the VAD. Specifically, the practitioner can attach the inner connector 12 of the syringe 16 to the VAD. The practitioner can then expel fluid from the syringe barrel 20 to the VAD by moving the plunger or stopper 22 through the syringe barrel 20.

### Connector Assembly Including Snap-Fit Lock

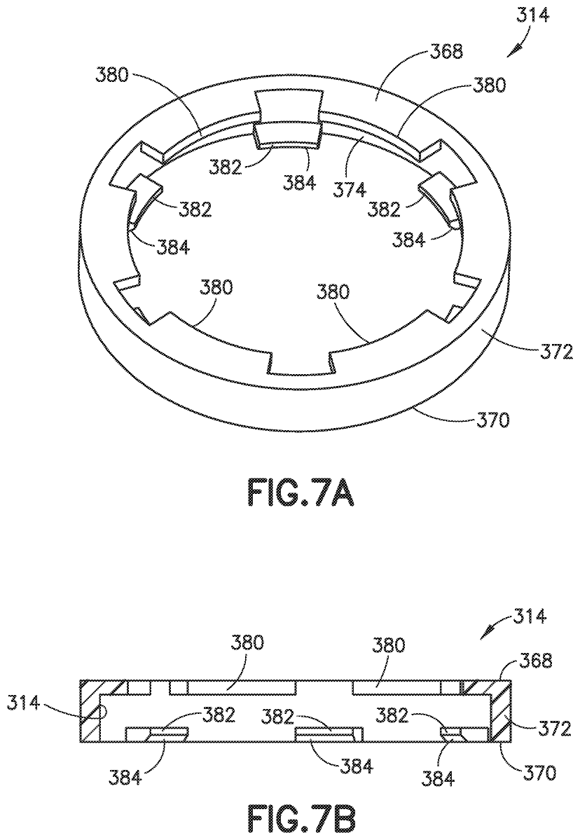

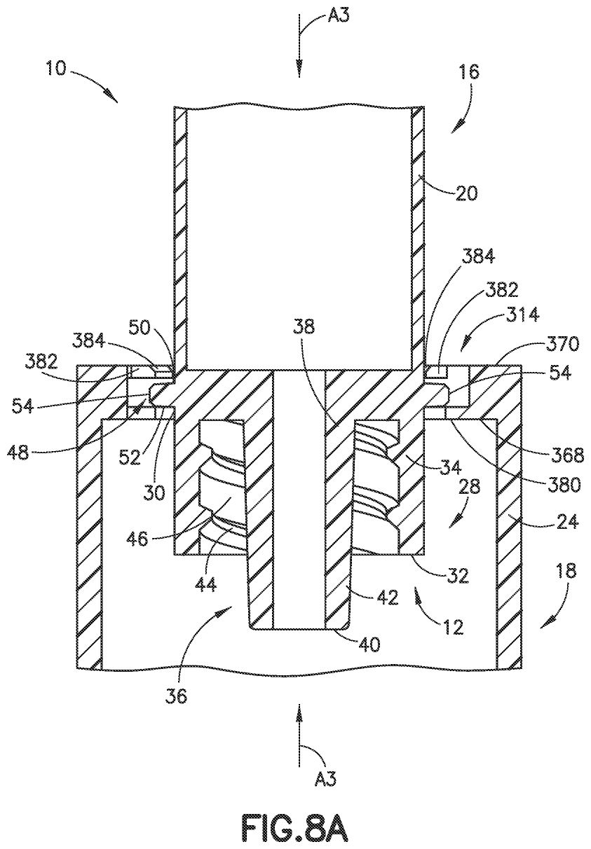

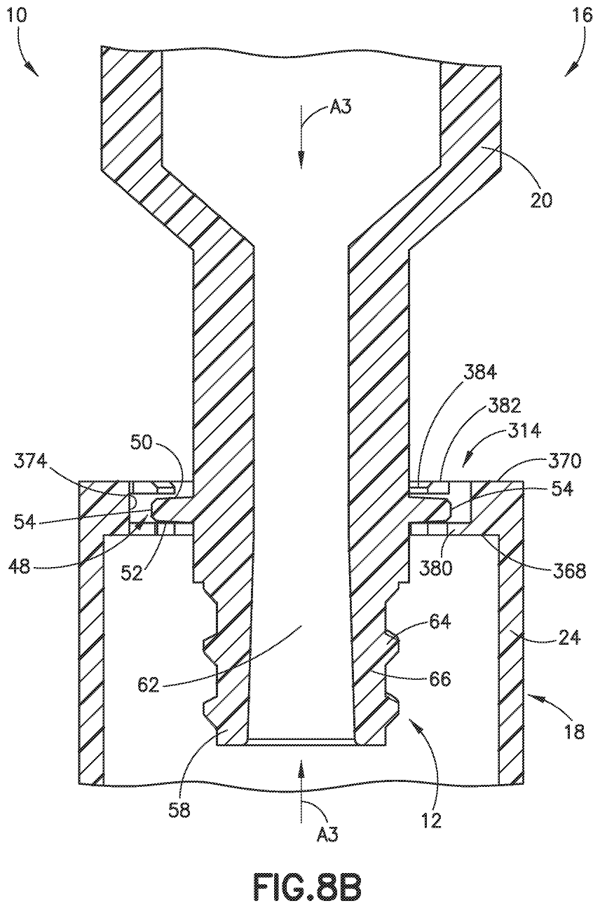

FIGS. 7A-8B show another example of an outer connector 314 that can be connected to the previously described inner connectors 12, such as the inner connector 12 including male luer components (shown in FIGS. 2A-2C) and/or to the inner connector 12 including female components (shown in FIGS. 3A-3C). More specifically, as in previous examples, the outer connector 314 is configured to be inserted over the inner connector 12 forming a releasable engagement between the inner connector 12 and the outer connector 314.

In some examples, the outer connector 314 can be an annular body including a first or proximal end 368, a second or distal end 370, and an annular sidewall 372 extending between the proximal end 368 and the distal end 370. The outer connector 314 also includes the protrusion, latch, lock, or similar engagement structure extending radially inwardly from an inner surface 374 of the sidewall 372 configured to engage the flange 48 of the inner connector 12 to secure the inner connector 12 to the outer connector 314.

Unlike in previous examples, however, the protrusion, lock, or latch of the outer connector 314 forms a snap-fit engagement with the flange 48, rather than the rotational or threaded engagement of previous examples described in connection with FIGS. 5A and 5B.

More specifically, as shown in FIGS. 7A and 7B, the lock or latch can be a snap-fit annular body or ring comprising snap tabs configured to engage surfaces 50, 52 of the flange 48. For example, the outer connector 314 can include first or proximal tabs 380 extending radially inwardly from the proximal end 368 of the sidewall 372 and distal tabs 382 extending radially inwardly from the distal end 370 of the sidewall 372. The flange 48 is configured to be inserted into the outer connector 314 through the distal end 370 and past the distal tabs 382. Therefore, as shown in FIGS. 7A and 7B, the distal tabs 382 can include a chamfered edge 384 to facilitate insertion of the flange 48 past the distal tabs 382. The proximal tabs 380 do not include the chamfered edge. The proximal tabs 380 and the distal tabs 382 are configured to retain the flange 48 of the inner connector 12 by a snap-fit engagement within a space defined by the proximal tabs 380 and the distal tabs 382. Accordingly, a distance between inner surfaces of the proximal tabs 380 and inner surfaces of the distal tabs 382 can correspond to a thickness of the flange 48 which, as previously described, can be about 0.2 mm to about 1.0 mm.

The outer connector 314 can have a variety of different arrangements of tabs 380, 382 depending, for example, on the design of the flange 48 and/or on how securely the flange 48 needs to be retained between the tabs 380, 382. For example, as shown in FIGS. 7A and 7B, the outer connector 314 includes six proximal tabs 380 and six distal tabs 382. In other examples, the outer connector 314 can have fewer than six tabs 380, 382 or more than six tabs 380, 382. The tabs 380, 382 are equidistantly spaced about the periphery of the outer connector 314. Further, as shown in FIGS. 7A and 7B, the proximal tabs 380 are axially offset from the distal tabs 382, such that the proximal tabs 380 do not overlap or cover the distal tabs 382. However, in other examples, the proximal tabs 380 can completely or partially overlap the distal tabs 382. Also, as shown in FIGS. 7A and 7B, an arcuate length of the proximal tabs 380 is longer than an arcuate length of the distal tabs 382, so that the distal tabs 382 can more easily bend or deform when inserting the flange 48 into the outer connector 314 past the distal tabs 382. In other examples, the distal tabs 382 can be thinner and/or more flexible than the proximal tabs 380, or can include bend points, notches, or living hinges so that the distal tabs 382 can be easily bent out of the away to accommodate insertion of the flange 48 into the outer connector 314.

FIGS. 8A and 8B are cross-sectional views showing a syringe 16 including the inner connector 12 connected to a disinfecting cap 18 including the outer connector 314. As shown in FIG. 8A, the inner connector 12 includes features of the male luer connector, such as the stem 36 and shield 28. The flange 48 is retained within the snap ring of the outer connector 314 between the proximal tabs 380 and the distal tabs 382. Specifically, the distal surface 52 of the flange 48 rests against an inwardly facing surface of the proximal tab 380, preventing the flange 48 from being moved farther into the disinfecting cap 18 or through the outer connector 314.

FIG. 8B shows an inner connector 12 including female luer components connected to the outer connector 314. As previously described, the flange 48 is retained within the outer connector 314 between the proximal tabs 380 and the distal tabs 382, with the distal surface 52 of the flange 48 resting against the inwardly facing surface of the proximal tab 380.

In order to connect the inner connector 12 to the outer connector 314, the practitioner moves the syringe 16 and inner connector 12 towards the outer connector 314, as shown by arrows A3 in FIGS. 8A and 8B, causing the flange 48 to contact the distal tabs 382 of the snap-ring of the outer connector 314. Due to the chamfered shape or chamfered surface 384 of the distal tabs 382, the flange 48 causes the distal tabs 382 to deform or deflect away from the flange 48 allowing the flange 48 to move past the distal tabs 382 and into the outer connector 314. The practitioner continues to move the inner connector 12 axially relative to the outer connector 314 causing the distal surface 52 of the flange 48 to contact the proximal tabs 380, as shown in FIGS. 8A and 8B. In this position, the flange 48 is engaged to the outer connector 314 forming a secure and fluid-tight connection between the inner connector 12 and the outer connector 314.

In order to remove the inner connector 12 from the outer connector 314, the practitioner can grasp the syringe 16 in one hand and the disinfecting cap 18 in the other hand and pull the syringe 16 away from the disinfecting cap 18. When sufficient force is applied to the syringe 16 and disinfecting cap 18, the flange 48 presses against the distal tabs 382 causing the distal tabs 382 to deform or deflect away from the flange 48. Once the distal tabs 382 are deflected or deformed away from the flange 48, the practitioner can pull the inner connector 12 away from the outer connector 314, thereby releasing the syringe 16 from the disinfecting cap 18.

While examples of the connector, connector assemblies, interfaces, systems, and methods of the present disclosure are shown in the accompanying figures and described hereinabove in detail, other examples will be apparent to, and readily made by, those skilled in the art without departing from the scope and spirit of the invention. Accordingly, the foregoing description is intended to be illustrative rather than restrictive. The invention described hereinabove is defined by the appended claims and all changes to the invention that fall within the meaning and the range of equivalency of the claims are to be embraced within their scope.

The invention claimed is:

1. A connector assembly comprising:
an inner connector comprising a first end, a second end, a sidewall extending between the first end and the second end, and a flange connected to or extending radially outward from the sidewall; and
an outer connector configured to be inserted over the inner connector comprising a first end, a second end, a sidewall extending therebetween, and a lock extending radially inwardly from an inner surface of the sidewall configured to engage the flange of the inner connector, thereby securing the inner connector to the outer connector.

2. The connector assembly of claim 1, wherein the inner connector comprises a male luer connector comprising:
a stem comprising a proximal end connected to the sidewall, a distal end protruding distally beyond the second end of the sidewall, and a body extending between the proximal end and the distal end through a space defined by the sidewall of the inner connector; and
at least one thread on an inner surface of the sidewall configured to engage corresponding threads of a female luer connector.

3. The connector assembly of claim 2, wherein the outer connector is configured to engage the inner connector without contacting the stem or the at least one thread of the inner connector.

4. The connector assembly of claim 1, wherein the inner connector is a female luer connector, and wherein the sidewall of the inner connector comprises threads on an outer surface of the sidewall configured to engage corresponding threads of a shield of a male luer connector.

5. The connector assembly of claim 4, wherein the outer connector is configured to engage the inner connector without contacting the threads on the outer surface of the sidewall of the inner connector.

6. The connector assembly of claim 1, wherein the inner connector comprises a vascular access device and the outer connector comprises an accessory device configured to engage the vascular access device.

7. The connector assembly of claim 6, wherein the vascular access device comprises at least one of a syringe, an infusion pump, a transfusion device, an intravenous drip device, or a catheter, and wherein the accessory device comprises a disinfecting cap, a safety device, a smart device, and/or a sensor device.

8. The connector assembly of claim 1, wherein the flange connected to or extending radially outward from the sidewall of the inner connector comprises a peripheral edge comprising opposing flat portions and opposing curved portions between the flat portions.

9. The connector assembly of claim 1, wherein the lock comprises a thread extending radially inwardly from at least a portion of the inner surface of the sidewall of the outer connector.

10. The connector assembly of claim 9, wherein the thread extends about the inner surface of the sidewall of the outer connector by less than a full rotation.

11. The connector assembly of claim 1, wherein the flange of the inner connector comprises a peripheral edge comprising opposing flat portions and opposing curved portions between the flat portions,
wherein the lock comprises a thread extending radially inwardly from at least a portion of the inner surface of the sidewall of the outer connector, and
wherein the outer connector further comprises at least one blocking tab separate from the thread extending radially inward from the sidewall of the outer connector disposed at the first end of the outer connector configured to contact the opposing curved portions of the flange to block further advancement of the inner connector into the outer connector.

12. The connector assembly of claim 1, wherein the lock comprises a locking ring comprising an annular sidewall, a plurality of first tabs extending radially inwardly from a first annular edge of the sidewall and a plurality of second tabs extending radially inwardly from a second annular edge of the sidewall.

13. The connector assembly of claim 12, wherein the plurality of first tabs and the plurality of second tabs are configured to retain the flange of the inner connector by a snap-fit engagement.

14. The connector assembly of claim 12, wherein the plurality of second tabs comprise chamfered surfaces for facilitating insertion of the inner connector into the outer connector.

15. The connector assembly of claim 12, wherein the flange is inserted into the locking ring by applying an axially directed force to the inner connector sufficient to deflect the plurality of second tabs away from the flange so that the flange moves into a space between the first tabs and the second tabs, and
wherein the flange is removed from the locking ring by applying a sufficient axial force to the inner connector or to the outer connector to overcome the snap-fit engagement between the locking ring and the flange so that the inner connector can be moved axially relative to the outer connector.

16. A vascular access device configured to be connected to an accessory device, the vascular access device comprising:
a tubular body defining a fluid path extending at least partially through the tubular body, the fluid path comprising a proximal end portion and a distal end portion;
a male connector connected to the tubular body comprising:
a stem defining a fluid path fluidly connected to the distal end portion of the fluid path of the tubular body configured to be inserted into a fluid path of a female connector;
a shield connected to a first end of the stem and at least partially enclosing the stem; and
a thread extending radially inward from an inner surface of the shield configured to engage a corresponding thread on an outer surface of the female connector; and
a flange connected to or extending radially outward from a sidewall of the shield configured to engage a lock extending radially inward from an inner surface of a tubular sidewall of the accessory device.

17. The vascular access device of claim 16, wherein the accessory device is configured to engage the flange of the vascular access device without contacting the stem or the at least one thread.

18. The vascular access device of claim 16, wherein the vascular access device comprises at least one of a syringe, an infusion pump, a transfusion device, an intravenous drip device, or a catheter, and wherein the accessory device comprises a disinfecting cap, a safety device, a smart device, and/or a sensor device.

19. An attachment method for attaching an inner connector to an outer connector, comprising:
inserting an inner connector comprising a first end, a second end, a sidewall extending between the first end and the second end, and a flange connected to or extending radially outward from the sidewall, into an outer connector comprising a lock extending radially inwardly from an inner surface of the outer connector; and
moving the inner connector through the outer connector, thereby causing the flange of the inner connector to engage the lock of the outer connector to secure the inner connector to the outer connector.

20. The method of claim 19, wherein the inner connector comprises a vascular access device and the outer connector comprises an accessory device configured to engage the vascular access device.
